(19) United States
(12) Patent Application Publication
(10) Pub. No.: US 2009/0192628 A1
(43) Pub. Date: Jul. 30, 2009

(54) Bone Substitute Material, Medical Material Comprising the Bone Substitute Material and Method for Manufacturing the Bone Substitute Material

(76) Inventors: Seishiro Ito, Ikoma-shi Nara (JP); Mitsunobu Iwasaki, Nara-shi Nara (JP)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/065,860

(22) PCT Filed: Aug. 31, 2006
(86) PCT No.: PCT/JP2006/317274

§ 371 Date: Mar. 18, 2009

(30) Foreign Application Priority Data
Sep. 8, 2005 (JP) 2005-261082

(51) Int. Cl. A61F 2/28; C25D 21/00; C25D 7/00

(52) U.S. Cl. 623/23.61; 205/98

(57) ABSTRACT

The object of the present invention is to provide a bone substitute material, medical material containing the bone substitute material and process for producing the bone substitute material wherein the bone material has excellent mechanical strength, biological affinity and biological activity.

The present invention provides a bone substitute material comprising a titanium or a titanium alloy and an anodic oxide film of the titanium or titanium alloy, wherein the anodic oxide film is formed on a surface of the titanium or titanium alloy, wherein inorganic compound microparticles are firmly fixed to a surface and/or inside of the anodic oxide film, and wherein the inorganic compound contains at least phosphorus and calcium. The present invention further provides a medical material comprising the bone substitute material and a method for manufacturing the bone substitute material.

### CROSS REFERENCE TO RELATED APPLICATION

This application is the National Stage of International Application No. PCT/JP2006/317274, filed Aug. 31, 2006, which claims priority of Japanese Application No. 2005-261082, filed Sep. 8, 2005, the entire disclosures of the preceding applications are incorporated by reference herein in its entirety.

### FIELD OF THE INVENTION

The present invention relates to a bone substitute material, a medical material comprising the bone substitute material and a method for manufacturing the bone substitute material. More specifically, it relates to the bone substitute material comprising a titanium or a titanium alloy, and an anodic oxide film of the titanium or titanium alloy, wherein the anodic oxide film is formed on a surface of the titanium or titanium alloy, and inorganic compound microparticles are firmly fixed to a surface or inside of the anodic oxide film, and the inorganic compound contains at least phosphorus and calcium.

### DESCRIPTION OF THE BACKGROUND ART

Currently, an autogeneous bone or an allogeneic bone from another person is transplanted to the patient when a bone is defected by a bone fracture, bone destruction or bone degeneracy. However, an artificial bone is used in case that the autogeneous bone is too small to cover the defective part, or that the allogeneic bone is not available. Various metals are used in the artificial bone. In particular, titanium and a titanium alloy are generally used as the bone substitute material, because they do not react specifically with biological body when they are placed in vivo as well as they have lightweight, nontoxic, and excellent mechanical property. However, BioMetal such as titanium does not combine directly to the bone and does not show bioactivity, while it has good biocompatibility, high corrosion resistance and toughness.

To solve this problem, bioactive hydroxyapatite has been physically-coated on the surface of titanium material by plasma spraying or lazer ablation method. Alternatively, a titanium oxide film having absorbed phosphate ion on its surface of the titanium material has been formed by anodizing titanium material, which results in having a biological affinity to the surface of the titanium material.

For example, Japanese patent publication 2003-190272 discloses that the titanium oxide film is formed on the surface of titanium material by an anodic oxidation, in which the film provides the material with the biological affinity. In Japanese patent publication 2004-531305, it is disclosed that a titanium oxide film, which contains calcium and phosphoric acid, is formed on the surface of titanium by anodic oxidation in an alkaline bath, and the formed film improves the biological affinity of the material. Japanese patent publication 2005-508862 discloses materials are treated with the anodic oxidation of titanium to provide the material with the titanium oxide film containing additive components such as calcium, phosphorus or sulfur.

However, materials, which are coated by bioactive hydroxyapatite on the surface of the titanium material by plasma spraying, have defects such as low adhesiveness between titanium and hydroxyapatite, and have difficulty in forming a uniform film on the entire surface of the complicated shape of the titanium material. Moreover, according to the method described in the patent document, the titanium is not anodized under the condition of spark discharge to obtain the titanium oxide film containing the inorganic compound, and this causes the film to be so thin. Thus, such a film has no sufficient biological affinity as well as less mechanical property.

### SUMMARY OF INVENTION

Considering the above problems, the inventors of the present invention found the method for forming a titanium oxide film having a thickness which can provide a sufficient mechanical strength to the surface of titanium or titanium alloy, and fixing inorganic compound microparticles containing phosphorus and calcium to the surface and/or inside of the film.

More specifically, one object of the present invention is to provide a bone substitute material and a medical material comprising the bone substitute material, which has an excellent mechanical strength as well as excellent biological affinity and biological activity. Another object of the present invention is to provide the method of manufacturing the bone substitute material which has an excellent mechanical strength as well as an excellent biological affinity and biological activity.

One embodiment of the present invention is related to a bone substitute material comprising a titanium or a titanium alloy, and an anodic oxide film of the titanium or titanium alloy, wherein the anodic oxide film is formed on a surface of the titanium or titanium alloy, wherein a pore part is formed on the surface of the anodic oxide film, wherein the pore part has an opening diameter of 0.1 μm to 10 μm, wherein calcium phosphate microparticles having a diameter of 10 nm to 10 μm are dispersively and firmly fixed to a surface and inside of the anodic oxide film, wherein the calcium phosphate is at least one selected from apatite fluoride, tricalcium phosphate (Ca₃(PO₄)₂) or calcium pyrophosphate (Ca₂P₂O₇).

Another embodiment of the present invention is related to a bone substitute material comprising a titanium or a titanium alloy, and an anodic oxide film of the titanium or titanium alloy, wherein the anodic oxide film is formed on a surface of the titanium or titanium alloy, wherein a pore part is formed on the surface of the anodic oxide film, wherein the pore part has an opening diameter of 0.1 μm to 10 μm, wherein calcium phosphate microparticles having a diameter of 10 nm to 10 μm are dispersively and firmly fixed to a surface and inside of the anodic oxide film, wherein the calcium phosphate is at least one selected from apatite fluoride, tricalcium phosphate (Ca₃(PO₄)₂) or calcium pyrophosphate (Ca₂P₂O₇).

Yet another embodiment of the present invention is related to the bone substitute material, wherein the anodic oxide film has a film thickness of 1 to 100 μm.

Yet another embodiment of the present invention is related to the bone substitute material, wherein the anodic oxide film has at least one crystal structure of titanium oxide selected from amorphous, rutile or anatase.

Yet another embodiment of the present invention is related to a medical material comprising the bone substitute material.

Yet another embodiment of the present invention is related to the bone substitute material, wherein the anodic oxide film has at least one crystal structure of titanium oxide selected from amorphous, rutile or anatase.

Yet another embodiment of the present invention is related to a medical material comprising the bone substitute material.

Yet another embodiment of the present invention is related to a method for manufacturing a bone substitute material, wherein the bone substitute material comprises a titanium or titanium alloy, and an anodic oxide film of the titanium or titanium alloy, wherein the anodic oxide film is formed on a surface of the titanium or titanium alloy, wherein calcium phosphate microparticles are dispersively and firmly fixed to a surface and inside of the anodic oxide film, comprising the steps of: (1) dispersing at least calcium phosphate microparticles in an electrolytic bath (2) anodizing a titanium or titanium alloy in the electrolytic bath obtained from the step (1), wherein the calcium phosphate microparticles are at least one selected from hydroxyapatite, apatite fluoride, tricalcium phosphate (Ca₃(PO₄)₂) or calcium pyrophosphate (Ca₂P₂O₇).

Yet another embodiment of the present invention is related to a method for manufacturing a bone substitute material, wherein the bone substitute material comprises a titanium or titanium alloy, and an anodic oxide film of the titanium or titanium alloy, wherein the anodic oxide film is formed on a surface of the titanium or titanium alloy, wherein calcium phosphate microparticles having a diameter of 10 nm to 10 μm are dispersively and firmly fixed to a surface and inside of the anodic oxide film, comprising the steps of: (1) dispersing at least calcium phosphate microparticles in an electrolytic bath (2) anodizing a titanium or titanium alloy in the electrolytic bath obtained from the step (1), wherein the electrolytic bath is an alkaline electrolytic bath comprising alkali metal hydroxide and/or alkali earth metal hydroxide, phosphate and complexing agent, wherein the calcium phosphate microparticles are at least one selected from hydroxyapatite, apatite fluoride, tricalcium phosphate (Ca₃(PO₄)₂) or calcium pyrophosphate (Ca₂P₂O₇), wherein the anodizing step in the step (2) is carried out under the condition of current density of 0.1 to 5 A/dm².

Yet another embodiment of the present invention is related to the method for manufacturing the bone substitute material, wherein the anodizing step in the step (2) is carried out under the condition of spark discharge

Yet another embodiment of the present invention is related to the method for manufacturing the bone substitute material, wherein the phosphate comprises one selected from orthophosphate ion, phosphoric hydrogen ion, dihydrogenphosphate ion or pyrophosphate ion and one selected from alkali metal ion, alkali earth metal ion or ammonium ion.

Yet another embodiment of the present invention is related to the method for manufacturing the bone substitute material, wherein the anodizing step in the step (2) is carried out under the condition of voltage of 80 to 300V.

Yet another embodiment of the present invention is related to the method for manufacturing the bone substitute material, wherein the anodizing step in the step (2) is carried out at the temperature of an electrolytic bath of 0 to 100 degrees.

Yet another embodiment of the present invention is related to the method for manufacturing the bone substitute material, wherein the phosphate comprises one selected from orthophosphate ion, phosphoric hydrogen ion, dihydrogenphosphate ion or pyrophosphate ion and one selected from alkali metal ion, alkali earth metal ion or ammonium ion.

Yet another embodiment of the present invention is related to the method for manufacturing the bone substitute material, wherein the anodizing step in the step (2) is carried out under the condition of voltage of 80 to 300V.

Yet another embodiment of the present invention is related to the method for manufacturing the bone substitute material, wherein the anodizing step in the step (2) is carried out at the temperature of an electrolytic bath of 0 to 100 degrees.

### BRIEF DESCRIPTIONS OF THE DRAWING

FIG. 1 is a diagrammatic perspective view showing the bone substitute material of the present invention. (However, microparticles of the inorganic compound of the present invention are not shown.)

FIG. 2 is a cross-section diagram showing the bone substitute material of the present invention. (However, microparticles of the inorganic compound of the present invention are not shown.)

FIG. 3 is cross-section diagrams of the bone substitute material of the present invention.

FIG. 4 shows X-ray diffraction pattern figures of Example 2 and Comparative example 1 comprising a XRD pattern of titanium (a), a XRD pattern of Comparative example 1(b), and a XRD pattern Example 2 (c). In the Fig., “R” means rutile, “A” means anatase, “T” means “titanium” and “HAp” means hydroxyapatite.

FIG. 5 shows X-ray diffraction pattern figures of Example 2, and the pattern figures shows the ones after being immersed in the SBF solution for 0 day, 3 days and 5 days respectively.

FIG. 6 shows SEM photographs of Example 2 and Comparative example 1. In the FIG. 6, (a) is a SEM photograph of an anodized titanium plate of Comparative example 1 before being immersed in the SBF solution, (b) is a SEM photograph of an anodized titanium plate of Comparative example 1 after being immersed in the SBF solution for 5 days, (c) is a SEM photograph of an anodized titanium plate of Comparative example 2 before being immersed in the SBF solution, and (d) is a SEM photograph of an anodized titanium plate of Comparative example 1 after being immersed in the SBF solution for 5 days.

FIG. 7 is a diagram showing precipitation amount of HAp microparticles per unit area in Test 2 “Time-course test of precipitation amount of HAp after being immersed in the SBF solution”.

FIG. 8 is a diagram showing the increasing amount of HAp film thickness per unit area in Test 2 “Time-course test of precipitation amount of HAp after being immersed in the SBF solution”.

### DETAILED DESCRIPTION OF THE INVENTION

The bone substitute material and the medical material comprising the bone substitute material of the present invention have an excellent mechanical strength as well as excellent biological affinity and biological activity.

More specifically, the bone substitute material of the present invention excels in biocompatibility and in adhesive property to biomedical tissue, and these advantages are derived from the inorganic compound including phosphorus and calcium. This is because inorganic compound microparticles containing phosphorus and calcium are firmly fixed to the surface of and/or within the bone substitute material of the present invention. In particular, because the inorganic compound microparticles according to the present invention are dispersed within the surface and/or inside of the anodic oxide film and firmly fixed to the surface and/or inside of the anodic oxide film, the materials according to present invention has stabilized biocompatibility and exhibits biocompatibility and in adhesive property to biomedical tissue uniformly or evenly.

The bone substitute material of the present invention has a sufficient thickness of the anodic oxide film formed on the surface of titanium and titanium alloy. It also has a high strength of the anodic oxide film. Further the anodic oxide film has a good adhesive property to the titanium material as well as excellent mechanical property. Therefore no abrasion or avulsion occurs in the bone substitute material.

The bone substitute material of the present invention is porous so that a biological bone and the artificial bone are strongly bonded to the biological bone.

As shown in one embodiment of the specification, once the bone substitute material of the present invention is immersed in SBF (simulated body fruid), the amount of HAp particles precipitated on and firmly fixed to the anodic oxide film is increased as the immersed-days passed. This clearly shows that the bone substitute material of the present invention has excellent biological activity.

The method for manufacturing the bone substitute material of the present invention can manufacture the bone substitute material having the excellent mechanical strength as well as excellent biological affinity and biological activity. In particular, the method for manufacturing the bone substitute material of the present invention is carried out easily because it only requires an electrolytic treatment to the titanium or the titanium alloy after dispersing the inorganic compound microparticles having biological affinity in the electrolytic bath.

Thus, the bone substitute material and the medical material comprising the bone substitute material achieved a high QOL (quality of life) of a patient such as reduction of days wearing a cast or improvement of the freedom in daily life.

A bone substitute material of the present invention and a medical material comprising the bone substitute material will be explained as below.

The bone substitute material of the present invention comprises at least one selected from titanium or titanium alloy, and an anodic oxide film of the titanium or titanium alloy.

The titanium alloy is not limited to, but the titanium alloy having lightweight, nontoxic and excellent mechanical property is preferably used. The shape of the titanium or titanium alloy may be determined in accordance with its usage.

The anodic oxide film of the titanium or titanium alloy is formed on a surface of the titanium or titanium alloy, and inorganic compound microparticles are firmly fixed to the surface and/or inside of the anodic oxide film. In addition, the inorganic compound microparticles contain at least phosphorus and calcium.

The titanium or titanium alloy may be covered with the oxide film of the titanium or titanium alloy in any size of area of its surface, but such a size may be determined in accordance with its usage, a type of the titanium or titanium alloy, and a type of inorganic compound microparticles (described below).

As the inorganic compound containing at least phosphorus and calcium, for example, calcium phosphate is used, and hydroxyapatite, apatite fluoride, tricalcium phosphate (Ca₃(PO₄)₂) and/or calcium pyrophosphate (Ca₂P₂O₇) are preferably used.

The inorganic compound preferably has the microparticle diameter of 10 nm to 10 μm, and more preferably 50 nm to 1 μm. This is because inorganic compound microparticles having the microparticle diameter of less than 10 nm may not be sufficiently and firmly fixed to the anodic oxide, and inorganic compound microparticles having the microparticle diameter of more than 10 μm may easily come off the anodic oxide film.

The bone substitute material of the present invention will be explained as below by referring to Figs.

The bone substitute material of the present invention comprises titanium or titanium alloy having a surface covered with an anodic oxide film.

The anodic oxide film is preferably porous, thus has multiple pore parts on its surface. The pore parts offer advantageous effect on the material. The pore parts not only increase the surface area to which inorganic compound microparticles are firmly fixed, but also provide anchor effect between the material and the biological bone to which the material is bound.

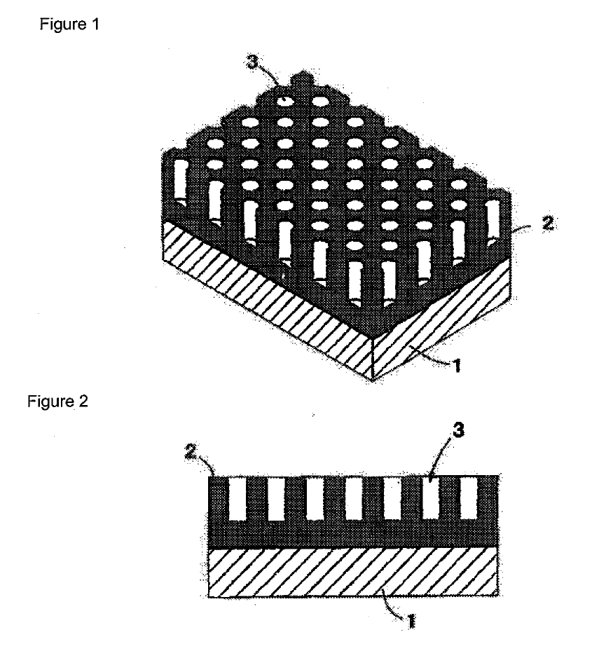

FIG. 1 shows a physical appearance of the titanium or titanium alloy having a surface coated with an anodic oxide film. FIG. 1 also shows titanium or titanium (1), an anodic oxide film (2) and a pore part (3). FIG. 2 is a cross-section diagram of FIG. 1. FIG. 2, like FIG. 1, shows titanium or titanium (1), an anodic oxide film (2) and a pore part (3). However, microparticles of the inorganic compound according to the present invention are not shown in FIG. 1 or 2 in order to clearly show the structures of the anodic oxide films. In addition, in FIGS. 1 to 3, the anodic oxide film is formed on the one side of the surface of the titanium or titanium alloy, but it may be formed on any side. For example, the anodic oxide film may be formed on the entire surface of the titanium or titanium alloy.

The anodic oxide film of the present invention may preferably have the film thickness of 1 to 100 μm, and more preferably 10 to 50 μm. The fixed inorganic compound microparticles may easily come off the film and the film may not have enough mechanical strength when the film thickness is less than 1 μm. Alternatively the anodic oxide film may have less adhesive ability to the titanium or titanium alloy plate when the film thickness is more than 100 μm.

For example, the anodic oxide film having the film thickness of 1 μm to 100 μm (i.e., sufficient film thickness unlike a thin film such as interference membrane) advantageously provides the anodic oxide film itself with sufficient mechanical strength. This is because such film thickness (i.e., 1 μm to 100 μm) may keep the basic titanium plate from being exposed after the film undergoes scratch test and is damaged. When the film thickness is more than 50 μm, the film has excellent mechanical strength. Alternatively when the film thickness is ranged from 10 to 50 μm, such a film thickness is also appropriate, and provides the anodic oxide film itself with sufficient mechanical strength. This results in creating sufficient adhesive ability to bind the anodic oxide film with the titanium or titanium alloy plate. Further such a thickness does not cause the film to come off the titanium or titanium alloy plate.

An opening diameter of the pore part on the anodic oxide film of the bone substitute material according to the present invention is preferably ranged from 0.1 to 10 μm, and more preferably 0.5 to 5 μm. The inorganic compound microparticles may not be fixed firmly to the pore part when the opening diameter is less than 0.1 μm, and the anodic oxide film itself may have less mechanical strength when the opening diameter is more than 10 μm.

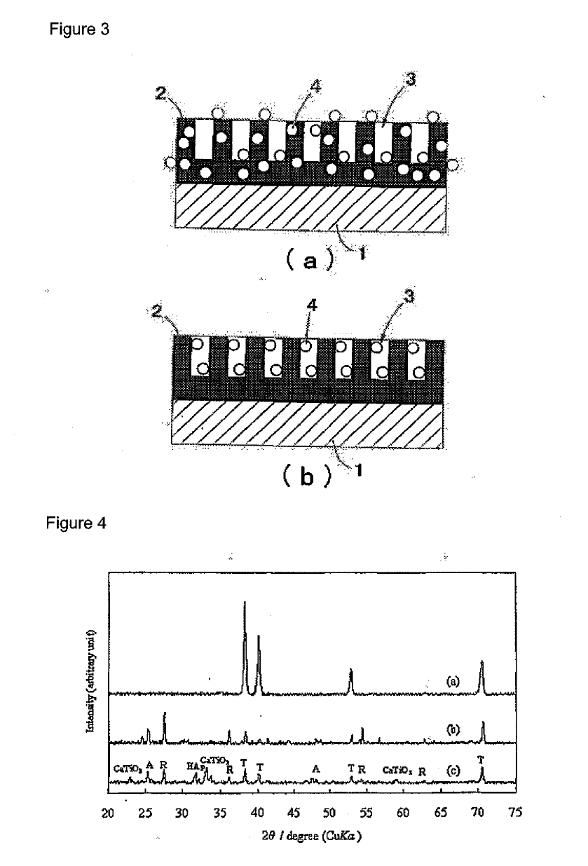

Next, FIG. 3 is referred. FIG. 3(a) and (b) show the bone substitute material of the present invention, more specifically, the material in which a titanium or titanium alloy have the surface covered with an oxide film, and this film is provided with a surface and/or inside to which inorganic compound microparticles are firmly fixed.

FIG. 3(a) shows the appearance in which inorganic compound microparticles are firmly fixed to the surface and/or inside of the anodic oxide film. Especially, the bone substitute material of the present invention has great and excellent bioactivity when inorganic compound microparticles are firmly fixed to both of the surface and inside of the anodic oxide film. This is because the inorganic compound microparticles promote to bond the material with a biological bone.

FIG. 3(b) shows the bone substitute material of the present invention in which inorganic compound microparticles are regularly and firmly fixed to a surface, especially to the inner side of a pore part formed on the anodic oxide film. When inorganic compound microparticles are firmly fixed to the inside of the pore part with regularity, the material offers excellent anchor effect between a bonded biological bone and the bone substitute material.

The crystal structure of the titanium oxide generated in the anodic oxide film according to the present invention is preferably at least one selected from amorphous, rutile or anatase. With these crystal structures, the bone substitute material excels in biocompatibility.

A medical material of the present invention comprises the bone substitute material of the present invention. The medical material of the present invention may be, for example, biomaterial, bone-filling material, denture material or protease.

Hereinafter, the method for manufacturing a bone substitute material of the present invention will be explained.

The method for manufacturing a bone substitute material of the present invention comprises the following steps (1) and (2).
(1) preparing a bath by dispersing inorganic compound microparticles (containing at least phosphorus and calcium)in an electrolytic bath
(2) anodizing a titanium or titanium alloy in the electrolytic bath obtained from the step (1)

First, the step (1) is explained.

In the step (1), inorganic compound microparticles are dispersed in a bath.

The inorganic compound microparticles dispersed in the step (1) contains at least phosphorus and calcium, for example, calcium phosphate. As calcium phosphates, hydroxyapatite, apatite fluoride, tricalcium phosphate (Ca₃(PO₄)₂) and/or calcium pyrophosphate (Ca₂P₂O₇) may be preferably used. This is because the bone substitute material having these calcium phosphates significantly improves an adhesive ability effect between the bone substitute material of the present invention and the bonded biological bone.

The amount of the inorganic compound microparticles in the bath may be determined in accordance with volume of the bath, volume of titanium or titanium alloy, or a type of or an amount of a compound to be added in the bath.

The electrolytic bath used in the step (1) may be either an acidic electrolytic bath or an alkaline electrolytic bath.

The acidic electrolytic bath of the present invention includes at least phosphoric acid and a complexing agent.

As phosphoric acid, phosphoric acid including phosphate ion may be used. For example, orthophosphoric acid, pyrophosphoric acid, polyphosphoric acid, metaphosphoric acid or tripolyphosphate may be used. Among them, orthophosphoric acid, pyrophosphoric acid and metaphosphoric acid may be preferably used, and orthophosphoric acid may be more preferably used.

As the complexing agent, hydrogen peroxide, ketones, amines or glycols may be used, and preferably hydrogen peroxide may be used.

In the acidic electrolytic bath, phosphate and/or inorganic acid may be further added as additive agent.

As the phosphate, for example, phosphate comprising one selected from orthophosphate ion, phosphoric hydrogen ion, dihydrogenphosphate ion or pyrophosphate ion and one selected from alkali metal ion, alkali earth metal ion or ammonium ion may be used. Among these, alkali metal ion and ammonium ion are preferably comprised, and more preferably sodium ion may be comprised.

As the inorganic acid, nitric acid, sulfuric acid, hydrochloric acid or boric acid may be used, and preferably boric acid may be used.

The alkaline electrolytic bath of the present invention contains phosphate, a complexing agent and at least one selected from alkali metal hydroxide or alkali earth metal hydroxide.

As alkali metal hydroxide and/or alkali earth metal hydroxide, sodium hydroxide may be preferably used.

As phosphate, for example, phosphate comprising one selected from orthophosphate ion, phosphoric hydrogen ion, dihydrogenphosphate ion or pyrophosphate ion and one selected from alkali metal ion, alkali earth metal ion or ammonium ion may be used. Among these, preferably alkali metal ion and ammonium ion may be comprised, and more preferably sodium ion may be comprised.

As the complexing agent, hydrogen peroxide, ketones, amines and glycols may be used, and preferably hydrogen peroxide may be used.

The acidic electrolytic bath of the present invention preferably has pH of 5 or lower, and more preferably pH of 3 or lower. In contrast, the alkaline electrolytic bath has pH of 9 or higher, and preferably pH of 11 or higher. This is because the low conductivity, which is caused by the electrolytic having pH higher than 5 or lower than 9, prevent the anodic oxide thick film from being formed with spark discharge.

The concentration of phosphoric acid, phosphate and the complexing agent may be preferably adjusted as below.

When phosphoric acid is included in the acidic electrolytic bath or alkaline electrolytic bath, the concentration of phosphoric acid may be preferably ranged from 0.01 to 10 M, and more preferably 1 to 5 M. This is because the bath (having the concentration of phosphoric acid of less than 0.01M) may not create conductivity enough to generate spark discharge, and therefore may not provide a sufficient film thickness. Alternatively, when the concentration is more than 10M, the inorganic compound microparticles are dissolved in the electrolytic bath, and therefore the inorganic compound microparticles may not be sufficiently fixed to the inside of the anodic oxide film.

When the complexing agent is included in the acidic electrolytic bath or alkaline electrolytic bath, the concentration of the complexing agent is preferably ranged from 0.001 to 3M, and more preferably from 0.01 to 0.5M. The complexing agent, which has the concentration of less than 0.001 M, may cause less complexing ability and less sustainability of the bath life. Alternatively, the complexing agent having the concentration of more 3M may not exhibit further advancement of complexing ability.

When phosphate is included in the acidic electrolytic bath or alkaline electrolytic bath, the concentration of phosphate is preferably ranged from 0.001 to 5M, and more preferably from 0.01 to 0.5M. The reason is that the film thickness is not sufficient because spark discharge may not occur due to low conductivity of the electrolytic bath when the concentration is less than 0.001M, and the anodic oxide film may lack in uniformity in its thickness when the concentration is more than 5M.

When alkali metal hydroxide and/or alkali earth metal hydroxide is included in the acidic electrolytic bath or alkaline electrolytic bath, the concentration of alkali metal hydroxide and/or alkali earth metal hydroxide is preferably ranged from 0.01 to 5M, and more preferably from 0.1 to 1M. The film may not be provided with a sufficient thickness because spark discharge does not occur due to low conductivity of the electrolytic bath when bath have the concentration of alkali metal hydroxide and/or alkali earth metal hydroxide of less than 0.01M. Alternatively the film lacks in uniformity in its thickness when the concentration is more than 5M.

Next, the step (2) is explained.

The step (2) is the step of anodizing a titanium or titanium alloy in the electrolytic bath obtained from the step (1). An anodic oxide film is formed on the surface of titanium or titanium alloy by the anodic oxidation and the anodic oxidation may be carried out by using the titanium or titanium alloy as the anodic electrode, and by applying the voltage with direct current, in which the voltage is equal to or higher than the voltage to create a spark discharge. By this means, the bone substitute material of the present invention is manufactured.

The inorganic compound microparticles of the present invention are firmly fixed to the surface and/or inside of the anodic oxide film. In detail, the localized heat, which occurs at the same time of the generation of the spark, is given the film during the step of forming the anodic oxide film. The inorganic compound microparticles in the electrolytic bath enter into or are fixed to the parts given the heat.

The anodic oxidation is carried out by applying direct current, superimposed direct current on altering current or wave pulse, or by applying single-phase half-wave, three-phase half-wave and six-phase half-wave with a direct current power source of a thyristor system. With the above wave patterns, the anodic oxidation is carried out at an voltage enough to generate spark discharge or more voltage to generate spark discharge, and preferably at an applied voltage of 100V or higher, and more preferably of 150V or higher.

The current density for the anodic oxidation is preferably ranged from 0.1 to 5 A/dm², and more preferably from 0.5 to 3 A/dm². When the current density is less than 0.1 A/dm², the film may not increase in thickness enough, and the fixed inorganic compound microparticles may easily come off the film. Alternatively the anodic oxidation using current density of more than 5 A/dm² may not provide the film with a sufficient density, because the speed of forming the anodic oxide film may be too fast to form the film.

The voltage for the anodic oxidation is ranged from 80 to 300V, preferably from 100 to 250V, more preferably from 150 to 200V. The reason is that the film may not be formed completely because spark discharge does not occur enough when the voltage is less than 50V, and the film may not be provided with a sufficient density because the speed of forming the anodic oxide film is too fast to form the film when the current density is more than 300V.

The electrolysis time in the step (2) is ranged from 5 to 240 minutes, preferably from 30 to 120 minutes. The fixed inorganic compound microparticles may easily come off the film due to insufficient thickness of the film when the time is less than 5 minutes. Alternatively when the time is more than 240 minutes, adhesive strength between the titanium plate and the film and mechanical strength of the film may not be provided enough due to overthickness of the film.

The temperature of the electrolytic bath in the step (2) is preferably ranged from 0 to 100 degree centigrade, more preferably from 20 to 40 degree centigrade. When the temperature is ranged from 0 to 100 degree centigrade, the liquid contained in the electrolytic bath is relatively easily controlled.

### (1) EXAMPLES

The invention will be explained by presenting examples as below.

(Test 1) Test for Measuring Biocompatibility and Bioactivity of the Bone Substitute Materials of the Present Invention

### [Production Method of Example 1]

100 g of calcium pyrophosphate (Ca₂P₂O₇) was dispersed in 1L of H₃PO₄—H₂O₂ system electrolytic bath including H₃BO₃ and H₂SO₄ as additive agents. A pure titanium plate (3 cm×5 cm) was immersed in the bath. After pressurizing the bath to 200V with 2.0 A/dm² direct current—constant current electrolysis, the voltage was maintained with potentiostatic electrolysis instead of the current electrolysis. In the bath whose voltage is kept at 200V for 60 minutes and whose electrolytic bath temperature is kept at 17 to 31 degree centigrade, the plate was anodically-oxidized. As a counter electrode, titanium plate, which was the same as the test plate, was used, in which the counter electrode was arranged with a distance of 5.0 cm from the other electrode. After the above, the plate was washed with water and dried. On the pure titanium plate, anodic oxide film was observed, having film thickness of 55 μm on its surface. The obtained bone substitute material was designated as Example 1.

### [Production Method of Example 2]

10 g of fine particle hydroxyapatite was dispersed in a mixture electrolytic bath including 0.5 mol/L sodium hydrate, 0.05 mol/L trisodium phosphate and 0.05 mol/L hydrogen peroxide. A pure titanium plate (3 cm×5 cm) was immersed in the bath. By keeping voltage at 150V with direct current for 120 minutes at electrolytic bath temperature of 20-30 degree centigrade, the plate was anodically-oxidized. As a counter electrode, titanium plate, which was the same as the test plate, was used, in which the counter electrode was arranged with a distance of 5.0 cm from the other electrode. After the above, the plate was washed with water and dried. On the pure titanium plate, anodic oxide film was observed, having film thickness of 15 μm on its surface. The obtained bone substitute material was designated as Example 2.

### [Production Method of Comparative Example 1]

A titanium plate was obtained by the same process as Example 2 except for not adding hydroxyapatite into the electrolytic bath, so that an anodic oxide film was formed on the surface of the titanium plate. The obtained bone substitute material was designated as Comparative example 1.

### [Preparation of SBF Solution (SBF: Simulated Body Fluid)]

NaCl 7.996 g, NaHCO₃ 0.35 g, KCl 0.224 g, K₂HPO₄ 0.174 g, MgCl₂.6H₂O 0.305 g, 1M-HCl 40 ml, CaCl₂ 0.278 g, Na₂SO₄ 0.071 g and (CH₂OH)₃CNH₂ 6.057 g were used and sequentially dissolved in this order with 700 ml of distilled water. After adjusting pH to 7.40 with 1M-HCl, the solution was transferred to a measuring flask. Distilled water was added thereto to obtain 1000 ml of the solution, and the obtained solution was designated as SBF solution.

### [Test Method]

By using the SBF solution, bioactivity of Example 1, Example 2 and Comparative example 1 was examined.

After immersing the Example 1, Example 2 and Comparative example 1 in the SBF (Simulated Body Fluid) solution for 5 days, precipitation amount of microparticles per unit area was measured.

### [Result]

As a result of measuring precipitation amount of hydroxyapatite in Example 1, it was 0.22 mg/cm², and the amount in Example 2 was 0.12 mg/cm². The precipitation of hydroxyapatite in Comparative example 1 was hardly recognized. Therefore, it is shown that the bone substitute materials of the present invention (Example 1 and Example 2) have bioactivity as well as biocompatibility.

### [X-ray Analysis Diagram (XRD Pattern) (1)]

X-ray analysis diagrams (XRD pattern) of Example 2 and Comparative example 1 were measured. The XRD patterns were shown in FIG. 4. Precipitation of rutile and anatase was observed from the measured result of Comparative example 1. Meanwhile, in the measured result of the Example 2, a peak of Hap was observed together with these crystals. This clearly shows that the bone substitute material of the present invention (Example 2) has biocompatibility.

### [X-ray Snalysis Diagram (XRD Pattern) (2)]

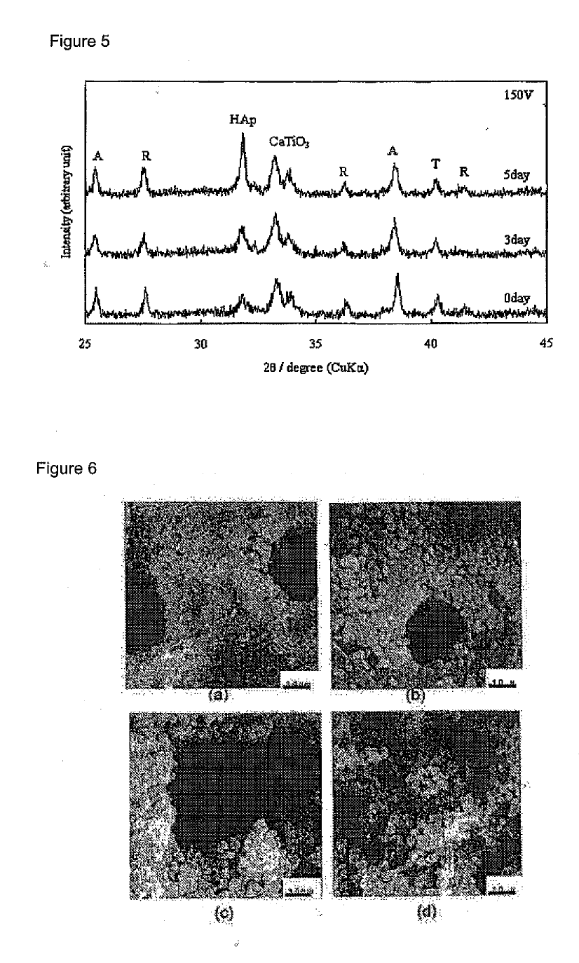

The XRD patterns of Example 2 after being immersed in the SBF solution for 0 day, 3 days and 5 days under the condition of maximum voltage of 150V were shown in FIG. 5. Peak intensity of HAp was significantly increased as the immersed-days passed. Thus, it is shown that the bone substitute material of the present invention has excellent bioactivity with biocompatibility.

In FIG. 6, (a) shows a SEM picture of Comparative example 1 before being immersed in the SBF solution, (b) shows a SEM picture of Comparative example 1 after being immersed in the SBF solution for 5 days, (c) shows a SEM picture of Example 2 before being immersed in the SBF solution, and (d) shows a SEM picture of Example 2 after being immersed in the SBF solution for 5 days. These SEM pictures clearly show that precipitation amount of HAp microparticles in the film of Example 2 after being immersed in the SBF solution for 5 days was increased as the immersed-days passed. Thus, it is shown that the bone substitute material of the present invention has bioactivity.

(Test2) Time-Course Test of Precipitation Amount of HAp After Being Immersed in the SBF Solution

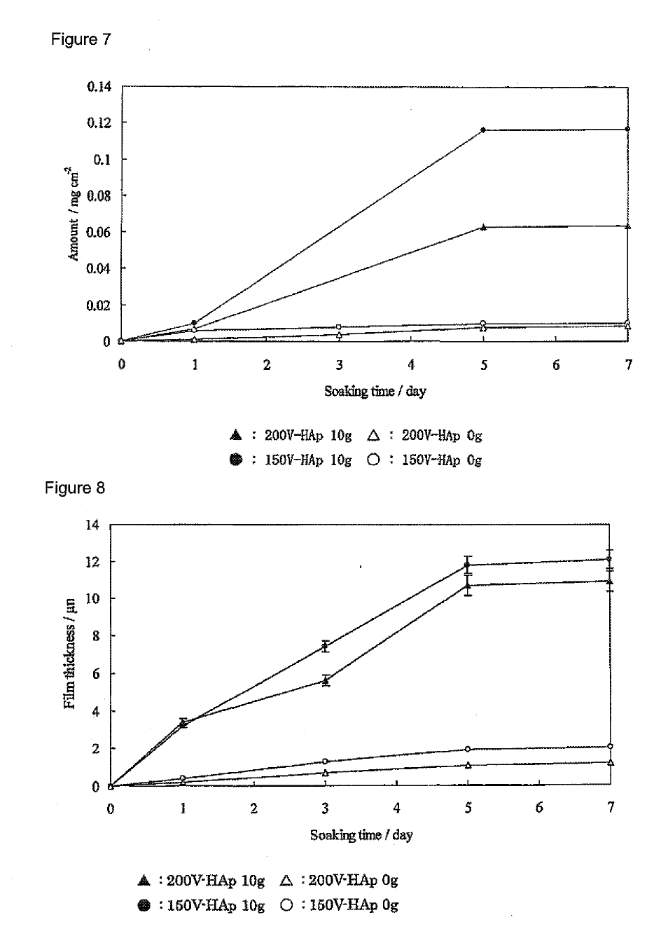

In the process of Example 2, an anodic oxide film material was prepared in 0.05M- Na₃PO₄-0.5M-NaOH-0.05M-H₂O₂ electrolytic bath on the condition of maximum voltage of 150V and of 200V. FIG. 7 and FIG. 8 show increased amounts of HAp precipitation amount per unit area and of HAp film thickness per unit area of the anodic oxide film material after being immersed in the SBF solution for 5 days respectively. Both of the HAp precipitation amount and the HAp film thickness were increased as the immersed-days passed.

The film anodically-oxidized under the condition of maximum voltage of 150V has a slightly bigger increasing amount of HAp precipitation amount and of HAp film thickness and higher bioactivity than those of 200V. In the XRD pattern of the anodically-oxidized film, the peak of anatase is bigger than the peak of rutile when the film is prepared under the condition of maximum voltage of 150V. Peak of anatase was almost same as that of rutile when the film is prepared under the condition of maximum voltage of 200V. Therefore, it is shown that anatase has higher apatite formation ability than rutile.

1-20. (canceled)

21. A bone substitute material comprising a titanium or a titanium alloy, and an anodic oxide film of the titanium or titanium alloy,
wherein the anodic oxide film is formed on a surface of the titanium or titanium alloy,
wherein calcium phosphate microparticles are dispersively and firmly fixed to a surface and inside of the anodic oxide film,
wherein the calcium phosphate is at least one selected from hydroxyapatite, apatite fluoride, tricalcium phosphate (Ca3(PO4)2) or calcium pyrophosphate (Ca2P2O7).

22. A bone substitute material comprising a titanium or a titanium alloy, and an anodic oxide film of the titanium or titanium alloy,
wherein the anodic oxide film is formed on a surface of the titanium or titanium alloy,
wherein a pore part is formed on the surface of the anodic oxide film,
wherein the pore part has an opening diameter of 0.1 μm to 10 μm,
wherein calcium phosphate microparticles having a diameter of 10 nm to 10 μm are dispersively and firmly fixed to a surface and inside of the anodic oxide film,
wherein the calcium phosphate is at least one selected from apatite fluoride, tricalcium phosphate (Ca3(PO4)2) or calcium pyrophosphate (Ca2P2O7).

23. The bone substitute material according to claim 21, wherein the anodic oxide film has a film thickness of 1 to 100 μm.

24. The bone substitute material according to claim 21, wherein the anodic oxide film has at least one crystal structure of titanium oxide selected from amorphous, rutile or anatase.

25. A medical material comprising the bone substitute material according to claim 21.

26. The bone substitute material according to claim 22, wherein the anodic oxide film has a film thickness of 1 to 100 μm.

27. The bone substitute material according to claim 22, wherein the anodic oxide film has at least one crystal structure of titanium oxide selected from amorphous, rutile or anatase.

28. A medical material comprising the bone substitute material according to claim 22.

29. A method for manufacturing a bone substitute material,
wherein the bone substitute material comprises a titanium or titanium alloy, and an anodic oxide film of the titanium or titanium alloy,
wherein the anodic oxide film is formed on a surface of the titanium or titanium alloy,
wherein calcium phosphate microparticles are dispersively and firmly fixed to a surface and inside of the anodic oxide film, comprising the steps of:
(1) dispersing at least calcium phosphate microparticles in an electrolytic bath
(2) anodizing a titanium or titanium alloy in the electrolytic bath obtained from the step (1)
wherein the calcium phosphate microparticles are at least one selected from hydroxyapatite, apatite fluoride, tricalcium phosphate (Ca3(PO4)2) or calcium pyrophosphate (Ca2P2O7).

30. A method for manufacturing a bone substitute material,
wherein the bone substitute material comprises a titanium or titanium alloy, and an anodic oxide film of the titanium or titanium alloy,
wherein the anodic oxide film is formed on a surface of the titanium or titanium alloy,
wherein calcium phosphate microparticles having a diameter of 10 nm to 10 μm are dispersively and firmly fixed to a surface and inside of the anodic oxide film, comprising the steps of:
(1) dispersing at least calcium phosphate microparticles in an electrolytic bath
(2) anodizing a titanium or titanium alloy in the electrolytic bath obtained from the step (1)
wherein the electrolytic bath is an alkaline electrolytic bath comprising alkali metal hydroxide and/or alkali earth metal hydroxide, phosphate and complexing agent,
wherein the calcium phosphate microparticles are at least one selected from hydroxyapatite, apatite fluoride, tricalcium phosphate (Ca3(PO4)2) or calcium pyrophosphate (Ca2P2O7)
wherein the anodizing step in the step (2) is carried out under the condition of current density of 0.1 to 5 A/dm2.

31. The method for manufacturing the bone substitute material according to claim 29, wherein the anodizing step in the step (2) is carried out under the condition of spark discharge.

32. The method for manufacturing the bone substitute material according to claim 29, wherein the phosphate comprises one selected from orthophosphate ion, phosphoric hydrogen ion, dihydrogenphosphate ion or pyrophosphate ion and one selected from alkali metal ion, alkali earth metal ion or ammoniumion.

33. The method for manufacturing the bone substitute material according to claim 29, wherein the anodizing step in the step (2) is carried out under the condition of voltage of 80 to 300V.

34. The method for manufacturing the bone substitute material according to claim 29, wherein the anodizing step in the step (2) is carried out at the temperature of an electrolytic bath of 0 to 100 degrees.

35. The method for manufacturing the bone substitute material according to claim 30, wherein the anodizing step in the step (2) is carried out under the condition of spark discharge.

36. The method for manufacturing the bone substitute material according to claim 30, wherein the phosphate comprises one selected from orthophosphate ion, phosphoric hydrogen ion, dihydrogenphosphate ion or pyrophosphate ion and one selected from alkali metal ion, alkali earth metal ion or ammonium ion.

37. The method for manufacturing the bone substitute material according to claim 30, wherein the anodizing step in the step (2) is carried out under the condition of voltage of 80 to 300V.

38. The method for manufacturing the bone substitute material according to claim 30, wherein the anodizing step in the step (2) is carried out at the temperature of an electrolytic bath of 0 to 100 degrees.
